(19) United States
(12) Patent Application Publication
(10) Pub. No.: US 2019/0209701 A1
(43) Pub. Date: Jul. 11, 2019

(54) COMBINATION THERAPY WITH A MEK INHIBITOR, A PD-1 AXIS INHIBITOR, AND A TAXANE

(71) Applicant: Genentech, Inc., South San Francisco, CA (US)

(72) Inventors: Nicholas Choong, Belmont, CA (US); Virginia McNally, Buckinghamshire (GB)

(21) Appl. No.: 16/366,411

(22) Filed: Mar. 27, 2019

Related U.S. Application Data
(63) Continuation of application No. PCT/US2017/053954, filed on Sep. 28, 2017.
(60) Provisional application No. 62/401,638, filed on Sep. 29, 2016.

(51) Int. Cl. A61K 47/64; A61K 31/4523; A61K 31/337; A61K 47/69; C07K 16/28; A61K 39/395; A61P 35/00

(52) U.S. Cl. CPC A61K 47/643; A61K 31/4523; A61K 31/337; A61K 47/6921; A61K 2039/505; A61K 39/39566; A61P 35/00; C07K 2317/24; C07K 2317/56; C07K 16/2827

(57) ABSTRACT

A combination therapy comprising a MEK inhibitor, a PD-1 or PD-L1 inhibitor, and a taxane is provided for the treatment of cancer, such as triple negative breast cancer.

### CROSS REFERENCE TO RELATED APPLICATIONS

This application is a continuation of International Patent Application No. PCT/US2017/053954 filed on Sep. 28, 2017, which claims priority benefit of U.S. Provisional Patent Application Ser. No. 62/401,638 filed on Sep. 29, 2016, both of which are incorporated herein in their entirety.

### SEQUENCE LISTING

The instant application contains a Sequence Listing which has been submitted electronically in ASCII format and is hereby incorporated by reference in its entirety. Said ASCII copy, created on Mar. 25, 2019, is named ‘33988-174_SEQ_LIST-32142238.txt’ and is 30,209 bytes in size.

### FIELD OF THE INVENTION

The field of the disclosure relates generally to cancer therapy with a combination of a MEK inhibitor, a PD-1 axis inhibitor, and a taxane.

### BACKGROUND OF THE INVENTION

Globally, breast cancer is the most common invasive malignancy and the most common cause of cancer related mortality in women (Siegel R, DeSantis C, Virgo K et al., Cancer treatment and survivorship statistics, 2012. CA Cancer J Clin 2012; 62:220-41) with 5 year survival following metastatic diagnosis of approximately 15%. Approximately 180,000 women are diagnosed with breast cancer in the United States annually, of whom 40,000 will die of the disease (Jemal et al. 2008) and the lifetime probability of developing invasive breast cancer in the United States and Europe is one in eight (Sasieni P D, Shelton J, Ormiston Smith N, et al., What is the lifetime risk of developing cancer: the effect of adjusting for multiple primaries, Br J Cancer 2011; 105(3):460-5).

About 10% to 20% of metastatic breast cancer is metastatic triple negative breast cancer (mTNBC). mTNBC tests negative for hormone epidermal growth factor receptor 2 (HER-2), estrogen receptors (ER), and progesterone receptors (PR). Since the tumor cells lack the necessary receptors, common treatments like hormone therapy and drugs that target estrogen, progesterone, and HER-2 are generally ineffective.

mTNBC and mBC are generally considered incurable. While responses to chemotherapy are common with mTNBC, the responses are not durable and likely a result of development of resistance. mTNBC being the only type of mBC without a targeted therapy results in mTNBC being a disease of significant unmet need.

### BRIEF DESCRIPTION

The present disclosure provides a method of treating a subject having breast cancer. The method comprises administering to said subject a therapy comprising (i) a therapeutically effective amount of a MEK inhibitor, (ii) a therapeutically effective amount of a PD-1 axis inhibitor, and (iii) a therapeutically effective amount of a taxane.

The present disclosure further provides a method of treating a subject having breast cancer, the method comprising administering to said subject a therapy comprising the following. A therapeutically effective amount of cobimetinib or a pharmaceutically acceptable salt thereof, a therapeutically effective amount of a PD-L1 inhibitor and a therapeutically effective amount of a taxane. The PD-L1 inhibitor that is an antibody comprising: (a) a heavy chain comprising HVR-H1 sequence of GFTFSDSWIH (SEQ ID NO:24), HVR-H2 sequence of AWISPYGGSTYYADSVKG (SEQ ID NO:25), and HVR-H3 sequence of RHWPGGFDY (SEQ ID NO:12); and a light chain comprising HVR-L1 sequence of RASQDVSTAVA (SEQ ID NO:26), HVR-L2 sequence of SASFLYS (SEQ ID NO:27), and HVR-L3 sequence of QQYLYHPAT (SEQ ID NO:28), or (b) a heavy chain variable region comprising the amino acid sequence of EVQLVESGGGLVQPGGSLRLSCAASGFTFSDSWIHWVRQAPGKGLEWVAWISPYGGST YYADSVKGRFTISADTSKNTAYLQMNSLRAEDTAVYYCARRHWPGGFDYWGQGTLVT VSS (SEQ ID NO:7) and a light chain variable region comprising the amino acid sequence of DIQMTQSPSSLSASVGDRVTITCRASQDVSTAVAWYQQKPGKAPKLLIY SASFLY SGVPSRFSGSGSGTDFTLTISSLQPEDFATYYCQQYLYHPATFGQGTK VEIKR (SEQ ID NO:9).

The present disclosure further provides a kit for treating breast cancer in a human subject. The kit comprises a MEK inhibitor, a PD-1 axis inhibitor, a taxane and a package insert comprising instructions for using a therapeutically effective amount of the MEK inhibitor, a therapeutically effective amount of the PD-1 axis inhibitor and a therapeutically effective amount of the taxane for treating the subject.

The order of administration of the MEK inhibitor, the PD-1 axis inhibitor, and the taxane may be varied. In some aspects, when the PD-1 axis inhibitor and the taxane are administered on the same day, the PD-1 axis inhibitor is administered prior to the taxane. In another aspect, the taxane is administered prior to the MEK inhibitor. This staggered approach of administering the taxane before the MEK inhibitor can help take advantage of the mechanism of cell kill by the taxane and maximize synergy with the MEK inhibitor. In another embodiment, the taxane and the PD-1 axis inhibitor are administered prior to administration of the MEK inhibitor.

The present disclosure further provides a breast cancer therapy drug combination comprising: (i) a MEK inhibitor in a dose of from about 20 mg to about 100 mg, from about 40 mg to about 80 mg, or about 60 mg; (ii) a PD-1 axis inhibitor in a dose of from about 400 mg to about 1200 mg, from about 600 mg to about 1000 mg, from about 700 mg to about 900 mg, or about 840 mg; and (iii) a taxane in a dose of from about 50 mg/m² body surface area to about 200 mg/m² body surface area, from about 50 mg/m² body surface area to about 200 mg/m² body surface area, from about 50 mg/m² body surface area to about 150 mg/m² body surface area, from about 75 mg/m² body surface area to about 125 mg/m² body surface area, from about 75 mg/m² body surface area to about 100 mg/m² body surface area, about 80 mg/m² body surface area, or about 100 mg/m² body surface area.

In some aspects of the disclosure, the MEK inhibitor is cobimetinib or a pharmaceutically acceptable salt thereof, the PD-L1 inhibitor is atezolizumab, and the taxane is paclitaxel or nab-paclitaxel.

### BRIEF DESCRIPTION OF THE DRAWINGS

FIG. 1A shows the study schema and treatment cohort I of a clinical trial and

FIG. 1B shows the study schema and treatment cohorts II and III of the clinical trial.

### DETAILED DESCRIPTION

The present disclosure is directed to the treatment of breast cancer with the combination of a MEK inhibitor, a PD-1 axis inhibitor and a taxane, and more particularly to the combination of cobimetinib or a pharmaceutically acceptable salt thereof, atezolizumab and paclitaxel or nab-paclitaxel. In some aspects, the cancer is mBC. In some other aspects, the cancer is mTNBC.

It is believed that the simultaneous inhibition of MEK, inhibition of PD-1 axis, and triggering of apoptosis or cell division inhibition may potentially enhance the response to this chemo-immunotherapy regimen by down regulating immunosuppressive factors and increasing lymphocytic infiltration in addition to cell cycle arrest and MEK inhibition. It is yet further believed that MEK inhibition may reduce paclitaxel resistance. It is further believed that patients with breast cancer, including mBC and mTNBC, may have some intrinsic resistance to taxane treatment, and that subjects with breast cancer could benefit from a cobimetinib/paclitaxel combination.

### Definitions

As used herein, “metastatic triple negative breast cancer” (mTNBC) refers to breast cancer cells that test negative for hormone epidermal growth factor receptor 2 (HER-2), estrogen receptors (ER), and progesterone receptors (PR). Typically, a patient is diagnosed as having mTNBC if the patient tests HER2 negative and the status of ER/PR is less than 10% ER/PR. ASCO guidelines set the ER/PR status as less than 1%.

As used herein, the term “cancer” refers to or describes the physiological condition in mammals that is typically characterized by unregulated cell growth. A “tumor” comprises one or more cancerous cells.

As used herein, the terms “patient” and “subject” refer to animals such as mammals, including, but not limited to, primates (e.g., humans), cows, sheep, goats, horses, dogs, cats, rabbits, rats, mice and the like. In certain aspects, the patient or subject is a human.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, the phrase “therapeutically effective amount” refers to an amount of one or more drug compounds that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the overall response rate (ORR). A therapeutically effective amount herein may vary according to factors such as the disease state, age, sex, and weight of the patient, and the ability of the agent to elicit a desired response in the individual. A therapeutically effective amount is also one in which a toxic or detrimental effect of the treatment is outweighed by the therapeutically beneficial effect. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, and enhancing effect of another medication such as via targeting, delaying the progression of the disease, and/or prolonging survival. In the case of a cancer or a tumor, a therapeutically effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. A therapeutically effective amount can be administered in one or more administrations. For purposes of this disclosure, a therapeutically effective amount of drug, compound, or pharmaceutical composition is an amount sufficient to accomplish prophylactic or therapeutic treatment either directly or indirectly. As is understood in the clinical context, a therapeutically effective amount of a drug, compound, or pharmaceutical composition may or may not be achieved in combination with another drug, compound, or pharmaceutical composition. Thus, a therapeutically effective amount may be considered in the context of administering one or more therapeutic agents, and a single agent may be considered to be given in a therapeutically effective amount if, in combination with one or more other agents, a desirable result may be or is achieved.

As used herein, “in combination with” refers to administration of one treatment modality in addition to another treatment modality. As such, “in combination with” refers to administration of one treatment modality before, during, or after administration of the other treatment modality to the individual.

As used herein, the term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, “immunohistochemistry” (IHC) refers to the process of detecting antigens (e.g., proteins) in cells of a tissue section by exploiting the principle of antibodies binding specifically to antigens in biological tissues. Immunohistochemical staining may be used in the diagnosis of abnormal cells such as those found in cancerous tumors. Specific molecular markers are characteristic of particular cellular events such as proliferation or cell death (apoptosis). IHC may also be used to understand the distribution and localization of biomarkers and differentially expressed proteins in different parts of a biological tissue. Antibodies or antisera, such as polyclonal antisera and monoclonal antibodies specific for each marker, are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. In one visualization method, an antibody is conjugated to an enzyme, such as peroxidase, that can catalyze a colour-producing reaction (see immunoperoxidase staining). In another visualization method, the antibody can also be tagged to a fluorophore, such as fluorescein or rhodamine (see immunofluorescence). Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

As used herein, “anti-therapeutic antibody assessment” (ATA) refers to an immunogenicity evaluation using a risk-based immunogenicity strategy as detailed in Rosenberg A S, Worobec A S., A risk-based approach to immunogenicity concerns of therapeutic protein products, BioPharm Intl 2004; 17:34-42; and Koren E, Smith H W, Shores E, et al., Recommendations on risk-based strategies for detection and characterization of antibodies against biotechnology products, J Immuno Methods 2008; 333:1-9) to characterize ATA responses. Each reference is incorporated by reference herein in its entirety.

As used herein, Cₘₐₓ refers to maximum plasma concentration.

As used herein, Cₘᵢₙ refers to minimum plasma concentration.

As used herein “area under concentration curve” (AUC) refers to the area under a fitted plasma concentration versus time curve. AUC_{0-∞} refers to area under curve baseline-infinity. AUC_{0-T} is total exposure.

As used herein “Response Evaluation Criteria in Solid Tumors” (RECIST) v1.1 refers to tumor response criteria conventions as detailed by Eisenhauer, E A, et al., New response evaluation criteria in solid tumours: Revised RECIST guideline (version 1.1), Eur J Cancer 2009:45:228-247; by Topalian S L, et al., Safety, activity, and immune correlates of anti-PD-L1 antibody in cancer, N Engl J Med 2012:366:2443-54; and by Wolchok J D, et al., Guidelines for the evaluation of immune therapy activity in solid tumors: immune-related response criteria, Clin Can Res 2009; 15:7412-20. Each reference is incorporated by reference herein in its entirety.

As used herein “Immune-Modified RECIST” (irRC) refers to criteria derived from RECIST v1.1 conventions (Eisenhauer, E A, et al., (2009)) and immune response criteria as detailed by Nishino M, et al., Optimizing immune-related tumor response assessment: does reducing the number of lesions impact response assessment in melanoma patients treated with ipilimumab, J Immunother Can 2014; 2:17; and Nishino M, Giobbie-Hurder A, Gargano Metal., Developing a common language for tumor response to immunotherapy: immune-related response criteria using unidimensional measurements, Clin Can Res 2013; 19:3936-43. Each reference is incorporated by reference herein in its entirety.). Unless otherwise specified, RECIST v1.1 conventions apply.

As used herein “inhibit” refers to a decrease in the activity of the target enzyme, as compared to the activity of that enzyme in the absence of the inhibitor. In some aspects, the term “inhibit” means a decrease in activity of at least about 5%, at least about 10%, at least about 20%, at least about 25%, at least about 50%, at least about 60%, at least about 70%, at least about 80%, at least about 90%, or at least about 95%. In other aspects, inhibit means a decrease in activity of about 5% to about 25%, about 25% to about 50%, about 50% to about 75%, or about 75% to 100%. In some aspects, inhibit means a decrease in activity of about 95% to 100%, e.g., a decrease in activity of 95%, 96%, 97%, 98%, 99%, or 100%. Such decreases can be measured using a variety of techniques that would be recognizable by one of skill in the art.

As used herein, “progression free survival” (PFS) refers to the time from the treatment of the disease to the first occurrence of disease progression or relapse as determined by the investigator using RECIST v1.1.

As used herein, “overall survival” (OS) refers to the time from randomization to death from any cause.

As used herein, “partial response” (PR) refers to at least a 30% decrease in the sum of diameters of target lesions, taking as reference the baseline sum of diameters.

As used herein, “delaying the progression” of a disease means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein “sustained response” refers to the sustained effect on reducing tumor growth after cessation of a treatment. For example, the tumor size may remain to be the same or smaller as compared to the size at the beginning of the administration phase. In some aspects, the sustained response has a duration at least the same as the treatment duration, at least 1.5×, 2×, 2.5×, or 3× of the length of the treatment duration.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

As used herein, “complete response” (CR) refers to the disappearance of all target lesions. Any pathological lymph nodes (whether target or non-target) have a reducing in short axis to less than 10 mm.

As used herein, “progressive disease” (PD) refers to at least a 20% increase in the sum of diameters of target lesions, taking as reference the smallest sum on study (nadir), including baseline and an absolute increase of at least 5 mm.

As used herein, “stable disease” (SD) refers to neither sufficient shrinkage to qualify for PR nor sufficient increase to qualify for PD, taking as reference the smallest sum on study.

As used herein, “overall response rate” (ORR) refers to the rate of a PR or CR occurring after randomization and confirmed ≥28 days later as determined by the investigator using RECIST v1.1.

As used herein, “unconfirmed overall response rate” (ORR_uc) refers to the rate of a PR or CR occurring after randomization as determined by the investigator using RECIST v1.1 where confirmation is not required.

As used herein, “duration of response” (DOR) refers to the time from the first occurrence of a documented objective response to the time of relapse, as determined by the investigator using RECIST v1.1 or death from any cause during the study, whichever occurs first.

As used herein, “National Cancer Institute Common Terminology Criteria for Adverse Events” (NCI CTCAE) refers to Common Terminology Criteria for Adverse Effect, Version 4.0, published May 28, 2009 (v4.03: Jun. 14, 2010) by the U.S. Department of Health and Human Services, National Institutes of Health, National Cancer Institute (Incorporated by reference in its entirety).

As used herein, “Functional Assessment of Cancer Therapy General” (FACT-G) refers to a validated and reliable 27-item questionnaire comprised of four subscales that measure physical (7 items), social/family (7 items), emotional (6 items) and functional wellbeing (7 items), and is considered appropriate for use with patients with any form of cancer (Cella D F, Tulsky D S, Gray G, Sarafian B, Linn E, Bonomi A E et al., The Functional Assessment of Cancer Therapy scale: development and validation of the general measure, Journal of Clinical Oncology 1993; 11(3 Supp1.2):570-9; and Webster, K., Odom, L., Peterman, A., Lent, L., Cella, D., The Functional Assessment of Chronic Illness Therapy (FACIT) measurement system: Validation of version 4 of the core questionnaire, Quality of Life Research 1999, 8(7):604. Each reference is incorporated herein in its entirety). Patients assess how true each statement has been for them in the previous 7 days on a five-point scale (0, not at all; 1, a little bit; 2, somewhat; 3, quite a bit; 4, very much).

As used herein, the term “MEK inhibitor(s)” refers to a molecule that inhibits a MEK, such as the mitogen-activated protein kinase enzymes MEK1 (also known as MAP2K1), or MEK2 (also known as MAP2K2). A MEK inhibitor may be used to affect the MAPK/ERK pathway that may be over active in some cancers, such as breast cancer. MEK inhibitors have been extensively reviewed (S. Price, Putative Allosteric MEK1 and MEK 2 inhibitors, Expert Opin. Ther. Patents, 2008 18(6):603; J. I. Trujillo, MEK Inhibitors: a patent review 2008-2010, Expert Opin. Ther. Patents 2011 21(7):1045).

As used herein, the term “PD-1 axis inhibitor” or “binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis inhibitor includes a PD-1 inhibitor, a PD-L1 inhibitor, and a PD-L2 inhibitor.

As used herein, the term “PD-1 inhibitor” or “binding antagonist” refers to a molecule that decreases, blocks, inhibits, abrogates or interferes with signal transduction resulting from the interaction of PD-1 with one or more of its binding partners, such as PD-L1 and PD-L2. In some embodiments, the PD-1 inhibitor is a molecule that inhibits the binding of PD-1 to one or more of its binding partners. In a specific aspect, the PD-1 inhibitor inhibits the binding of PD-1 to PD-L1 and/or PD-L2. For example, PD-1 inhibitors include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 inhibitor reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 inhibitor is an anti-PD-1 antibody.

As used herein, the term “PD-L1 inhibitor” or “binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 inhibitor is a molecule that inhibits the binding of PD-L1 to its binding partners. In a specific aspect, the PD-L1 inhibitor inhibits binding of PD-L1 to PD-1 and/or B7-1. In some embodiments, the PD-L1 inhibitor include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 inhibitor reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 inhibitor is an anti-PD-L1 antibody.

As used herein, the term “PD-L2 inhibitor” or “binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 inhibitor is a molecule that inhibits the binding of PD-L2 to one or more of its binding partners. In a specific aspect, the PD-L2 inhibitor inhibits binding of PD-L2 to PD-1. In some embodiments, the PD-L2 inhibitor include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 inhibitor reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 inhibitor is an immunoadhesin.

As used herein, “taxane” refers to a diterpene which may bind to tubulin, promoting microtubule assembly and stabilization and/or prevent microtubule depolymerization, which results in the inhibition of mitosis in cells and concomitant triggering of apoptosis or reversion to cell cycle G-phase in the absence of cell division. Taxanes have been extensively reviewed (R. van Vuuren, Antimitotic drugs in the treatment of cancer, Cancer Chemother Pharmacol. 2015; 76; 1101-1112; I. Ojima, Taxane anticancer agents: a patent perspective, Expert Opin. Ther. Patents, 2016 18(6):1-20).

As used herein, the term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth. As used herein, the term “dysfunctional” also includes refractory or unresponsive to antigen recognition, specifically, impaired capacity to translate antigen recognition into down-stream T-cell effector functions, such as proliferation, cytokine production (e.g., IL-2) and/or target cell killing.

As used herein, the term “anergy” refers to the state of unresponsiveness to antigen stimulation resulting from incomplete or insufficient signals delivered through the T-cell receptor (e.g. increase in intracellular Ca+2 in the absence of ras-activation). T cell anergy can also result upon stimulation with antigen in the absence of co-stimulation, resulting in the cell becoming refractory to subsequent activation by the antigen even in the context of co-stimulation. The unresponsive state can often be overridden by the presence of Interleukin-2. Anergic T-cells do not undergo clonal expansion and/or acquire effector functions.

As used herein, the term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways (PD-1, B7-H3, B7-H4, etc.).

“Enhancing T-cell function” means to induce, cause or stimulate a T-cell to have a sustained or amplified biological function, or renew or reactivate exhausted or inactive T-cells. Examples of enhancing T-cell function include: increased secretion of gamma-interferon from CD8+ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, 200%. The manner of measuring this enhancement is known to one of ordinary skill in the art.

A “T cell dysfunctional disorder” is a disorder or condition of T-cells characterized by decreased responsiveness to antigenic stimulation. In a particular embodiment, a T-cell dysfunctional disorder is a disorder that is specifically associated with inappropriate increased signaling through PD-1. In another embodiment, a T-cell dysfunctional disorder is one in which T-cells are anergic or have decreased ability to secrete cytokines, proliferate, or execute cytolytic activity. In a specific aspect, the decreased responsiveness results in ineffective control of a pathogen or tumor expressing an immunogen. Examples of T cell dysfunctional disorders characterized by T-cell dysfunction include unresolved acute infection, chronic infection and tumor immunity.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“κ”) and lambda (“λ”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthiin, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the disclosure may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10-7 M, preferably no more than about 1×10-8 M and preferably no more than about 1×10-9 M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The expression “linear antibodies” refers to the antibodies described in Zapata et al. (1995 Protein Eng, 8(10):1057-1062). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA, and/or RNA), polynucleotide copy number alterations (e.g., DNA copy numbers), polypeptides, polypeptide and polynucleotide modifications (e.g. posttranslational modifications), carbohydrates, and/or glycolipid-based molecular markers.

As used herein, the term “package insert” refers to instructions customarily included in commercial packages of therapeutic products, that contain information about the indications, usage, dosage, administration, contraindications and/or warnings concerning the use of such therapeutic products.

The term “pharmaceutically acceptable salts” denotes salts which are not biologically or otherwise undesirable. Pharmaceutically acceptable salts include both acid and base addition salts. The phrase “pharmaceutically acceptable” indicates that the substance or composition must be compatible chemically and/or toxicologically, with the other ingredients comprising a formulation, and/or the mammal being treated therewith. Acid addition salts are formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid “mesylate”, ethanesulfonic acid, p-toluenesulfonic acid, and salicyclic acid. Base addition salts are formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethyl amine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

### Therapeutic Agents

The present disclosure uses the combination of a MEK inhibitor, a PD-1 axis inhibitor, and a taxane to treat breast cancer in a subject. In some aspects, the MEK inhibitor is cobimetinib or a pharmaceutically acceptable salt thereof; the PD-1 axis inhibitor is a PD-L1 inhibitor, and more particularly the PD-L1 inhibitor is atezolizumab; and/or, the taxane is paclitaxel or nab-paclitaxel. In some other aspects, cobimetinib is Cotellic®, atezolizumab is Tecentriq®, paclitaxel is TAXOL®, and/or nab-paclitaxel is ABRAXANE®.

The presently disclosed compounds may be administered in any suitable manner known in the art. In some aspects, the compounds may be administered intravenously, intramuscularly, subcutaneously, topically, orally, transdermally, intraperitoneally, intraorbitally, by implantation, by inhalation, intrathecally, intraventricularly, intratumorally, or intranasally.

It is understood that appropriate doses of the active compound depends upon a number of factors within the knowledge of the ordinarily skilled physician. The dose(s) of the active compound will vary, for example, depending upon the age, body weight, general health, gender, and diet of the subject, the time of administration, the route of administration, the rate of excretion, and any drug combination.

It will also be appreciated that the effective dosage of the compound of the present disclosure, or a pharmaceutically acceptable salts, prodrugs, metabolites, or derivatives thereof used for treatment may increase or decrease over the course of a particular treatment. Changes in dosage may result and become apparent from the results of diagnostic assays.

### MEK Inhibitors

Examples of MEK inhibitors within the scope of the present disclosure include cobimetinib, trametinib, binimetinib, selumetinib, pimasertinib, refametinib, PD-0325901 and BI-847325, and pharmaceutically acceptable salts thereof.

In some particular aspects of the disclosure, the MEK inhibitor is cobimetinib or a pharmaceutically acceptable salt thereof (e.g., Cotellic®). Cobimetinib is a reversible, potent, and highly selective inhibitor of MEK1 and MEK2 (central components of the RAS/RAF/MEK/ERK (MAPK)) pathway and has single agent anti-tumor activity in multiple human cancer models. Cobimetinib has the CAS Registry Number 1168091-68-6, is of the chemical name 5)[3,4-Difluoro-2-(2-fluoro-4-iodophenylamino)phenyl][3-hydroxy-3-(piperidin-2-yl]azetidin-1-yl) methanone, is of the below structure:

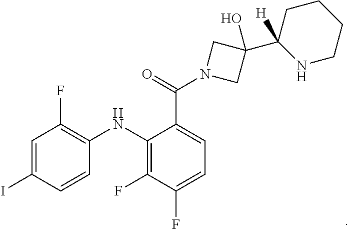 [SMILES: O=C(c1ccc(F)c(F)c1Nc1ccc(I)cc1F)N1CC(O)([C@@H]2CCCCN2)C1]

Cotellic® is the fumarate salt of cobimetinib. Cobimetinib is described in U.S. Pat. Nos. 7,803,839 and 8,362,002, each of which is incorporated by reference in its entirety.

Cobimetinib inhibits proliferation of a variety of human tumor cell lines through inhibition of MEK1 and MEK2. In addition, cobimetinib inhibits ERK phosphorylation in xenograft tumor models and stimulates apoptosis. Cobimetinib accumulates in tumor xenografts and remains at high concentrations in the tumor after plasma concentrations have declined. The activity of cobimetinib to inhibit ERK1 phosphorylation is more closely correlated with its concentration in tumor tissue than in plasma; in general, there is a good correlation between reduced ERK1 phosphorylation and efficacy in tumor xenograft models. Tumor regression has been observed in several human tumor xenograft models. This regression was dose dependent with up to 100% regression at the highest doses tested. The models studied include CRC, malignant melanoma, breast carcinoma, and lung carcinoma.

The pharmacokinetics of cobimetinib administered as a single agent have been characterized in cancer patients following oral administration after single and multiple dosing in Study MEK4592g which included evaluation of a cobimetinib dose of 60 mg per day in patients who harbored a BRAF, NRAS, or KRAS mutation. Overall 6 patients (all of whom had melanoma; 6.2%) had a confirmed partial response (PR), 28 patients (28.9%) had stable disease (SD), and 40 patients (41.2%) had progressive disease. Out of the 14 colorecatal cancer (CRC) patients, all patients experienced progressive disease (PD). In Stage III of Study MEK4592g, 18 patients were accrued, and best overall response was assessed for 14 of 18 patients. Four patients (22.2%) had SD as their best overall response, and 2 patients (11.1%) had unconfirmed tumor responses.

Cobimetinib has a moderate rate of absorption (median time to maximum concentration [tₘₐₓ] of 1 to 3 hours) and a mean terminal half-life (t_{1/2}) of 48.8 hours (a range of 23.1 to 80 hours). Cobimetinib binds to plasma proteins (95%) in a concentration-independent manner. Cobimetinib exhibits linear pharmacokinetics in the dose range of 0.05 mg/kg (approximately 3.5 mg/kg for 70 kg adult) to 80 mg and the absolute bioavailability was determined to be 45.9% (90% CI: 39.74%, 53.06%) in study MEK4952g in healthy subjects. Cobimetinib pharmacokinetics are not altered when administered in the fed state compared with administration in the fasted state in healthy subjects. Since food does not alter cobimetinib pharmacokinetics, cobimetinib can be administered with or without food. The proton pump inhibitor rabeprazole appears to have a minimal effect on cobimetinib pharmacokinetics, whether administered in the presence or absence of a high-fat meal compared with cobimetinib administration alone in the fasted state. Thus, increase in gastric pH does not affect cobimetinib pharmacokinetics, indicating it is not sensitive to alterations in gastric pH.

Cobimetinib salts, crystalline forms and prodrugs are within the scope of the present disclosure. Cobimetinib, preparative methods, and therapeutic uses are disclosed in International Publication Numbers WO 2007/044515, WO 2007/044615, WO 2014/027056 and WO 2014/059422, each of which is incorporated herein by reference. For instance, in some aspects of the present disclosure, the MEK inhibitor is crystalline hemifumarate cobimetinib polymorph Form A.

MEK inhibitor (e.g., cobimetinib) doses within the scope of the present disclosure are from about 20 mg to about 100 mg, from about 40 mg to about 80 mg, or about 60 mg of the MEK inhibitor per day. In particular embodiments, the MEK inhibitor is cobimetinib, and is dosed at about 60 mg, about 40 mg or about 20 mg.

The MEK inhibitor is suitably administered once daily. In some aspects, the MEK inhibitor is administered once daily for 21 consecutive days of a 28-day treatment cycle. In some aspects, the MEK inhibitor is administered once daily on days 1 to 21 or on days 3 to 23 of a 28-day treatment cycle.

### PD-1 Axis Inhibitors

In accordance with the present disclosure, a PD-1 axis inhibitor may more particularly refer to a PD-1 inhibitor, a PD-L1 inhibitor, or a PD-L2 inhibitor. Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, lambrolizumab, and CT-011. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

In some embodiments, the anti-PD-1 antibody is nivolumab (CAS Registry Number: 946414-94-4). In a still further embodiment, provided is an isolated anti-PD-1 antibody comprising a heavy chain variable region comprising the heavy chain variable region amino acid sequence from SEQ ID NO:1 and/or a light chain variable region comprising the light chain variable region amino acid sequence from SEQ ID NO:2. In a still further embodiment, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 1)

QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLEPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,

or
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 2)

EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some embodiments, the anti-PD-1 antibody is pembrolizumab (CAS Registry Number: 1374853-91-4). In a still further embodiment, provided is an isolated anti-PD-1 antibody comprising a heavy chain variable region comprising the heavy chain variable region amino acid sequence from SEQ ID NO:3 and/or a light chain variable region comprising the light chain variable region amino acid sequence from SEQ ID NO:4. In a still further embodiment, provided is an isolated anti-PD-1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 3)

QVQLVQSGVE VKKPGASVKV SCKASGYTFT NYYMYWVRQA
PGQGLEWMGG INPSNGGTNF NEKFKNRVTL TTDSSTTTAY
MELKSLQFDD TAVYYCARRDYRFDMGFDYW GQGTTVTVSS
ASTKGPSVFP LAPCSRSTSE STAALGCLVKDYFPEPVTVS
WNSGALTSGV HTFPAVLQSS GLYSLSSVVT
VPSSSLGTKTYTCNVDHKPS NTKVDKRVES KYGPPCPPCP
APEFLGGPSV FLFPPKPKDTLMISRTPEVT CVVVDVSQED
PEVQFNWYVD GVEVHNAKTK PREEQFNSTYRVVSVLTVLH
QDWLNGKEYK CKVSNKGLPS SIEKTISKAK
GQPREPQVYTLPPSQEEMTK NQVSLTCLVK GFYPSDIAVE
WESNGQPENN YKTTPPVLDSDGSFFLYSRL TVDKSRWQEG
NVFSCSVMHE ALHNHYTQKS LSLSLGK,

or
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 4)

EIVLTQSPAT LSLSPGERATLSCRASKGVSTSGYSYLHWYQQKPGQAP
RLLIYLASYLESGVPARFSGSGSGTDFTLTISSLEPEDFAVYYCQHSRD
LPLTFGGGTKVEIKRTVAAPSVFIFPPSDEQLKSGTASVVCLLNNFYPR
EAKVQWKVDNALQSGNSQESVTEQDSKDSTYSLSSTLTLSKADYEK
HKVYACEVTHQGLSSPVTKSFNRGEC.

In some embodiments, the PD-L1 inhibitor is anti-PD-L1 antibody. In some embodiments, the anti-PD-L1 inhibitor is selected from the group consisting of YW243.55.570, MPDL3280A (atezolizumab), MDX-1105, and MEDI4736. MDX-1105, also known as BMS-936559, is an anti-PD-L1 antibody described in WO2007/005874. Antibody YW243.55.570 (heavy and light chain variable region sequences shown in SEQ ID Nos. 5 and 6, respectively) is an anti-PD-L1 described in WO 2010/077634 A1. MEDI4736 is an anti-PD-L1 antibody described in WO2011/066389 and US2013/034559.

Examples of anti-PD-L1 antibodies useful for the methods herein, and methods for making thereof are described in PCT patent application WO 2010/077634 A1 and U.S. Pat. No. 8,217,149, which are incorporated herein by reference.

In some embodiments, the PD-1 axis inhibitor is an anti-PD-L1 antibody. In some embodiments, the anti-PD-L1 antibody is capable of inhibiting binding between PD-L1 and PD-1 and/or between PD-L1 and B7-1. In some embodiments, the anti-PD-L1 antibody is a monoclonal antibody. In some embodiments, the anti-PD-L1 antibody is an antibody fragment selected from the group consisting of Fab, Fab′-SH, Fv, scFv, and (Fab′)₂ fragments. In some embodiments, the anti-PD-L1 antibody is a humanized antibody. In some embodiments, the anti-PD-L1 antibody is a human antibody.

The anti-PD-L1 antibodies useful herein, including compositions containing such antibodies, such as those described in WO 2010/077634 A1. In some embodiments, the anti-PD-L1 antibody comprises a heavy chain variable region comprising the amino acid sequence of SEQ ID NO:7 or 8 (Infra) and a light chain variable region comprising the amino acid sequence of SEQ ID NO:9 (Infra).

In one embodiment, the anti-PD-L1 antibody contains a heavy chain variable region polypeptide comprising an HVR-H1, HVR-H2 and HVR-H3 sequence, wherein:

(SEQ ID NO: 10)

(a) the HVR-H1 sequence is GFTFSX₁SWIH;

(SEQ ID NO: 11)

(b) the HVR-H2 sequence is AWIX₂PYGGSX₃YYADSVKG;

(SEQ ID NO: 12)

(c) the HVR-H3 sequence is RHWPGGFDY;

further wherein: X₁ is D or G; X₂ is S or L; X₃ is T or S.

In one specific aspect, X₁ is D; X₂ is S and X₃ is T. In another aspect, the polypeptide further comprises variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4). In yet another aspect, the framework sequences are derived from human consensus framework sequences. In a further aspect, the framework sequences are VH subgroup III consensus framework. In a still further aspect, at least one of the framework sequences is the following:

(SEQ ID NO: 13)

HC-FR1 is EVQLVESGGGLVQPGGSLRLSCAAS

(SEQ ID NO: 14)

HC-FR2 is WVRQAPGKGLEWV

(SEQ ID NO: 15)

HC-FR3 is RFTISADTSKNTAYLQMNSLRAEDTAVYYCAR

(SEQ ID NO: 16)

HC-FR4 is WGQGTLVTVSA.

In a still further aspect, the heavy chain polypeptide is further combined with a variable region light chain comprising an HVR-L1, HVR-L2 and HVR-L3, wherein:

(SEQ ID NO: 17)

(a) the HVR-L1 sequence is RASQX₄X₅X₆TX₇X₈A;

(SEQ ID NO: 18)

(b) the HVR-L2 sequence is SASX₉LX₁₀S,;

(SEQ ID NO: 19)

(c) the HVR-L3 sequence is QQX₁₁X₁₂X₁₃X₁₄PX₁₅T;

further wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T.

In a still further aspect, X₄ is D; X₅ is V; X₆ is 5; X₇ is A; X₈ is V; X₉ is F; X₁₀ is Y; X₁₁ is Y; X₁₂ is L; X₁₃ is Y; X₁₄ is H; X₁₅ is A. In a still further aspect, the light chain further comprises variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the framework sequences are VL kappa I consensus framework. In a still further aspect, at least one of the framework sequence is the following:

(SEQ ID NO: 20)

LC-FR1 is DIQMTQSPSSLSASVGDRVTITC

(SEQ ID NO: 21)

LC-FR2 is WYQQKPGKAPKLLIY

(SEQ ID NO: 22)

LC-FR3 is GVPSRFSGSGSGTDFTLTISSLQPEDFATYYC

(SEQ ID NO: 23)

LC-FR4 is FGQGTKVEIKR.

In another embodiment, provided is an isolated anti-PD-L1 antibody or antigen binding fragment comprising a heavy chain and a light chain variable region sequence, wherein: the heavy chain comprises and HVR-H1, HVR-H2 and HVR-H3, wherein further:

(SEQ ID NO: 10)

(i) the HVR-H1 sequence is GFTFSX₁SWIH;

(SEQ ID NO: 11)

(ii) the HVR-H2 sequence is AWIX₂PYGGSX₃YYADSVKG

(SEQ ID NO: 12)

(iii) the HVR-H3 sequence is RHWPGGFDY, and

the light chain comprises and HVR-L1, HVR-L2 and HVR-L3, wherein further:

(SEQ ID NO: 17)

(i) the HVR-L1 sequence is RASQX₄X₅X₆TX₇X₈A

(SEQ ID NO: 18)

(ii) the HVR-L2 sequence is SASX₉LX₁₀S; and

(SEQ ID NO: 19)

(iii) the HVR-L3 sequence is QQX₁₁X₁₂X₁₃X₁₄PX₁₅T;.

further wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, F or I; X₁₄ is H, V, P, T or I; X₁₅ is A, W, R, P or T.

In a specific aspect, X₁ is D; X₂ is S and X₃ is T. In another aspect, X₄ is D; X₅ is V; X₆ 1 is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is 5; X₇ is A; X₈ is V; X₉ is F; X₁₀ is Y; is Y; X₁₂ is L; X₁₃ is Y; X₁₄ is H and X₁₅ is A.

In a further aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1

(SEQ ID NO: 13)

EVQLVESGGGLVQPGGSLRLSCAAS
HC-FR2

(SEQ ID NO: 14)

WVRQAPGKGLEWV
HC-FR3

(SEQ ID NO: 15)

RFTISADTSKNTAYLQMNSLRAEDTAVYYCAR
HC-FR4

(SEQ ID NO: 16)

WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1

(SEQ ID NO: 20)

DIQMTQSPSSLSASVGDRVTITC
LC-FR2

(SEQ ID NO: 21)

WYQQKPGKAPKLLIY
LC-FR3

(SEQ ID NO: 22)

GVPSRFSGSGSGTDFTLTISSLQPEDFATYYC
LC-FR4

(SEQ ID NO: 23)

FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect the minimal effector function results from an “effector-less Fc mutation” or aglycosylation. In still a further embodiment, the effector-less Fc mutation is an N297A or D265A/N297A substitution in the constant region.

In yet another embodiment, provided is an anti-PD-L1 antibody comprising a heavy chain and a light chain variable region sequence, wherein:
(a) the heavy chain further comprises and HVR-H1, HVR-H2 and an HVR-H3 sequence having at least 85% sequence identity to GFTFSDSWIH (SEQ ID NO:24), AWISPYGGSTYYADSVKG (SEQ ID NO:25) and RHWPGGFDY (SEQ ID NO:12), respectively, or
(b) the light chain further comprises an HVR-L1, HVR-L2 and an HVR-L3 sequence having at least 85% sequence identity to RASQDVSTAVA (SEQ ID NO:26), SASFLYS (SEQ ID NO:27) and QQYLYHPAT (SEQ ID NO:28), respectively.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1

(SEQ ID NO: 13)

EVQLVESGGGLVQPGGSLRLSCAAS
HC-FR2

(SEQ ID NO: 14)

WVRQAPGKGLEWV
HC-FR3

(SEQ ID NO: 15)

RFTISADTSKNTAYLQMNSLRAEDTAVYYCAR
HC-FR4

(SEQ ID NO: 16)

WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1

(SEQ ID NO: 20)

DIQMTQSPSSLSASVGDRVTITC
LC-FR2

(SEQ ID NO: 21)

WYQQKPGKAPKLLIY
LC-FR3

(SEQ ID NO: 22)

GVPSRFSGSGSGTDFTLTISSLQPEDFATYYC
LC-FR4

(SEQ ID NO: 23)

FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect the minimal effector function results from an “effector-less Fc mutation” or aglycosylation. In still a further embodiment, the effector-less Fc mutation is an N297A or D265A/N297A substitution in the constant region.

In a still further embodiment, provided is an isolated anti-PD-L1 antibody comprising a heavy chain and a light chain variable region sequence, wherein:
(a) the heavy chain sequence has at least 85% sequence identity to the heavy chain sequence:

(SEQ ID NO: 29)

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSA,

or
(b) the light chain sequence has at least 85% sequence identity to the light chain sequence:

(SEQ ID NO: 9)

DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1

(SEQ ID NO: 13)

EVQLVESGGGLVQPGGSLRLSCAAS
HC-FR2

(SEQ ID NO: 14)

WVRQAPGKGLEWV
HC-FR3

(SEQ ID NO: 15)

RFTISADTSKNTAYLQMNSLRAEDTAVYYCAR
HC-FR4

(SEQ ID NO: 16)

WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1

(SEQ ID NO: 20)

DIQMTQSPSSLSASVGDRVTITC
LC-FR2

(SEQ ID NO: 21)

WYQQKPGKAPKLLIY
LC-FR3

(SEQ ID NO: 22)

GVPSRFSGSGSGTDFTLTISSLQPEDFATYYC
LC-FR4

(SEQ ID NO: 23)

FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further embodiment, the effector-less Fc mutation is an N297A or D265A/N297A substitution in the constant region.

In another further embodiment, provided is an isolated anti-PD-L1 antibody comprising a heavy chain and a light chain variable region sequence, wherein:
(a) the heavy chain sequence has at least 85% sequence identity to the heavy chain sequence:

(SEQ ID NO: 7)

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,

or
(b) the light chain sequence has at least 85% sequence identity to the light chain sequence:

(SEQ ID NO: 9)

DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In a still further embodiment, provided is an isolated anti-PD-L1 antibody comprising a heavy chain and a light chain variable region sequence, wherein:
(a) the heavy chain sequence has at least 85% sequence identity to the heavy chain sequence:

(SEQ ID NO: 8)

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTK,

or
(b) the light chain sequences has at least 85% sequence identity to the light chain sequence:

(SEQ ID NO: 9)

DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1

(SEQ ID NO: 13)

EVQLVESGGGLVQPGGSLRLSCAAS
HC-FR2

(SEQ ID NO: 14)

WVRQAPGKGLEWV
HC-FR3

(SEQ ID NO: 15)

RFTISADTSKNTAYLQMNSLRAEDTAVYYCAR
HC-FR4

(SEQ ID NO: 30)

WGQGTLVTVSS.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1

(SEQ ID NO: 20)

DIQMTQSPSSLSASVGDRVTITC
LC-FR2

(SEQ ID NO: 21)

WYQQKPGKAPKLLIY
LC-FR3

(SEQ ID NO: 22)

GVPSRFSGSGSGTDFTLTISSLQPEDFATYYC
LC-FR4

(SEQ ID NO: 23)

FGQGTKVEIKR.

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further embodiment, the effector-less Fc mutation is an N297A or D265A/N297A substitution in the constant region.

In yet another embodiment, the anti-PD-L1 antibody is atezolizumab, or MPDL3280A (CAS Registry Number: 1422185-06-5). In a still further embodiment, provided is an isolated anti-PD-L1 antibody comprising a heavy chain variable region comprising the heavy chain variable region amino acid sequence from EVQLVESGGGLVQPGGSLRLSCAASGFTFSDSWIHWVRQAPGKGLEWVAWISPYGGST YYADSVKGRFTISADTSKNTAYLQMNSLRAEDTAVYYCARRHWPGGFDYWGQGTLVT VSS (SEQ ID NO:7) or EVQLVESGGGLVQPGGSLRLSCAASGFTFSDSWIHWVRQAPGKGLEWVAWI SPYGGSTYYADSVKGRFTISADTSKNTAYLQMNSLRAEDTAVYYCARRHWPGGFDYW GQGTLVTVSSASTK (SEQ ID NO:8) and a light chain variable region comprising the amino acid sequence of DIQMTQSPSSLSASVGDRVTITCRASQDVSTAVAWYQQKPGKAPKLLIY SASFLYSGVPSRFSGSGSGTDFTLTISSLQPEDFATYYCQQYLYHPATFGQGTKVEIKR (SEQ ID NO:9). In a still further embodiment, provided is an isolated anti-PD-L1 antibody comprising a heavy chain and/or a light chain sequence, wherein:
(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

(SEQ ID NO: 31)

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGEDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLEPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,

and/or
(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 32)

DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In a still further embodiment, provided is an isolated nucleic acid encoding a light chain or a heavy chain variable region sequence of an anti-PD-L1 antibody, wherein:
(a) the heavy chain further comprises and HVR-H1, HVR-H2 and an HVR-H3 sequence having at least 85% sequence identity to GFTFSDSWIH (SEQ ID NO:24), AWISPYGGSTYYADSVKG (SEQ ID NO:25) and RHWPGGFDY (SEQ ID NO:12), respectively, and
(b) the light chain further comprises an HVR-L1, HVR-L2 and an HVR-L3 sequence having at least 85% sequence identity to RASQDVSTAVA (SEQ ID NO:26), SASFLYS (SEQ ID NO:27) and QQYLYHPAT (SEQ ID NO:28), respectively.

In a specific aspect, the sequence identity is 86%, 87%, 88%, 89%, 90%, 91%, 92%, 93%, 94%, 95%, 96%, 97%, 98%, 99% or 100%. In one aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

HC-FR1

(SEQ ID NO: 13)

EVQLVESGGGLVQPGGSLRLSCAAS
HC-FR2

(SEQ ID NO: 14)

WVRQAPGKGLEWV
HC-FR3

(SEQ ID NO: 15)

RFTISADTSKNTAYLQMNSLRAEDTAVYYCAR
HC-FR4

(SEQ ID NO: 16)

WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

LC-FR1

(SEQ ID NO: 20)

DIQMTQSPSSLSASVGDRVTITC
LC-FR2

(SEQ ID NO: 21)

WYQQKPGKAPKLLIY
LC-FR3

(SEQ ID NO: 22)

GVPSRFSGSGSGTDFTLTISSLQPEDFATYYC
LC-FR4

(SEQ ID NO: 23)

FGQGTKVEIKR.

In a still further specific aspect, the antibody described herein (such as an anti-PD-1 antibody, an anti-PD-L1 antibody, or an anti-PD-L2 antibody) further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further aspect, the effector-less Fc mutation is an N297A or D265A/N297A substitution in the constant region.

In a still further aspect, provided herein are nucleic acids encoding any of the antibodies described herein. In some embodiments, the nucleic acid further comprises a vector suitable for expression of the nucleic acid encoding any of the previously described anti-PD-L1, anti-PD-1, or anti-PD-L2 antibodies. In a still further specific aspect, the vector further comprises a host cell suitable for expression of the nucleic acid. In a still further specific aspect, the host cell is a eukaryotic cell or a prokaryotic cell. In a still further specific aspect, the eukaryotic cell is a mammalian cell, such as Chinese Hamster Ovary (CHO).

The antibody or antigen binding fragment thereof, may be made using methods known in the art, for example, by a process comprising culturing a host cell containing nucleic acid encoding any of the previously described anti-PD-L1, anti-PD-1, or anti-PD-L2 antibodies or antigen-binding fragment in a form suitable for expression, under conditions suitable to produce such antibody or fragment, and recovering the antibody or fragment.

In some embodiments, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In this regard it is to be noted that the pharmacokinetics of atezolizumab administered as a single agent have been characterized based on clinical data from study PCD4989g and are consistent with a currently ongoing Phase III Study WO29522 in first line treatment of TNBC. Atezolizumab anti-tumor activity has been observed across doses from 1 to 20 mg/kg. Overall, atezolizumab exhibits pharmacokinetics that are both linear and consistent with typical IgG1 antibodies for doses ≥1 mg/kg every three weeks (q3w). Pharmacokinetic data (Bai S, Jorga K, Xin Y, et al., A guide to rational dosing of monoclonal antibodies, Clin Pharmacokinet 2012; 51:119-35, incorporated by reference herein in its entirety) does not suggest any clinically meaningful differences in exposure following a fixed dose or a dose adjusted for weight. Atezolizumab dosing schedules of q3w and q2w have been tested. A fixed dose of atezolizumab 800 mg every two weeks (q2w) (equivalent to a body weight-based dose of 10 mg/kg q2w) results in equivalent exposure to the Phase III dose of 1200 mg administered every three weeks (q3w). The q3w schedule is being used in multiple Phase III studies of atezolizumab monotherapy across multiple tumor types and the q2w predominantly used in combination with chemotherapy regimens. In Study PCD4989g, the Kaplan-Meier estimated overall 24-week progression-free survival (PFS) rate was 33% (95% CI: 12%, 53%).

The PD-1 axis inhibitor doses of the present disclosure are suitably from about 400 mg to about 1200 mg, from about 600 mg to about 1000 mg, from about 700 mg to about 900 mg, or about 840 mg. In some aspects, the PD-1 axis inhibitor is a PD-L1 inhibitor, and more particularly is atezolizumab, which is administered at a dose of about 840 mg.

In particular embodiments, the PD-1 axis inhibitor, or more particularly the PD-L1 inhibitor, is administered intravenously every 14 days of a 28-day treatment cycle. In some aspects, the subject is treated with the PD-1 axis inhibitor, and more particularly the PD-L1 inhibitor, on days 1 and 15 of the 28-day treatment cycle

### Taxanes

Examples of taxanes within the scope of the present disclosure include paclitaxel (i.e., TAXOL®, CAS #33069-62-4), nab-paclitaxel (i.e., ABRAXANE®, nanoparticle albumin-bound paclitaxel), docetaxel (i.e., TAXOTERE®, CAS #1 14977-28-5), larotaxel, cabazitaxel, milataxel, tesetaxel, and/or orataxel. In some aspects, the taxane is a prodrug form and/or conjugated form of taxane (e.g., DHA covalently conjugated to paclitaxel, paclitaxel poliglumex, and/or linoleyl carbonate-paclitaxel). In some particular aspects, the taxane is paclitaxel or nab-paclitaxel.

Taxane doses within the scope of the present disclosure are suitably from about 50 mg/m² to about 200 mg/m², from about 50 mg/m² to about 150 mg/m², from about 75 mg/m² to about 125 mg/m², or from about 75 mg/m² to about 100 mg/m², or about 80 mg/m² where m² refers to patient body surface area. In some aspects of the disclosure, taxane is dosed weekly for three weeks of a 28-day treatment cycle. In some aspects, the subject is treated with the taxane on days 1, 8 and 15 of the 28-day treatment cycle. In some aspects, the subject is treated weekly with about 80 mg/m² of paclitaxel. In some aspects, the subject is treated weekly with about 100 mg/m² of nab-paclitaxel. Calculation of body surface area for the purposes of dosing of paclitaxel should be made according to the prescribing information. In such aspects of the disclosure, paclitaxel will be administered as an infusion over a period of approximately 1 hour per standard practice or institutional guidelines. In some aspects of the disclosure, the patients receiving paclitaxel may be premedicated with dexamethasone, diphenhydramine, and an H2 blocker 30-60 minutes prior to the paclitaxel administration and according to the paclitaxel Package Insert and institutional guidelines.

### Breast Cancer

In one aspect, provided herein is a method for treating breast cancer in a subject in need thereof comprising administering to the subject a therapeutically effective amount of a combination of a MEK inhibitor, a PD-1 axis inhibitor, and a taxane. mBC and mTNBC are particularly amenable to the combination therapy described herein.

In some aspects of the disclosure, the treatment results in delaying the progression of the breast cancer in the subject. In some other aspects, the treatment results in a complete response in the subject. In some other aspects, the response is sustained after cessation of the treatment. In still other aspects, the treatment prolongs the median progression-free survival time as compared to a breast cancer, mBC or mTNBC subject receiving a therapy comprising (i) the therapeutically effective amount of the PD-1 axis inhibitor and the therapeutically effective amount of the MEK inhibitor and without administration of the taxane, (ii) the therapeutically effective amount the PD-1 axis inhibitor and the therapeutically effective amount of the taxane and without administration of the MEK inhibitor, and/or (iii) the therapeutically effective amount the MEK inhibitor and the therapeutically effective amount of the taxane and without administration of the PD-1 axis inhibitor.

### Combination Therapies

It is believed that the triple combination of a MEK inhibitor, a PD-1 axis inhibitor, and a taxane (i) targets the hallmarks of cancer (i.e., proliferative signaling, immune evasion, and cell cycle progression), (ii) will lead to synergistic anti-tumor activity based upon the complex interplay and activity these agents exhibit, and/or (iii) will offer the potential for substantial clinical benefit in patients with breast cancer, such as mBC or mTNBC. It is further believed that the triple combination of a MEK inhibitor, a PD-1 axis inhibitor, and a taxane may potentially enhance the response to this chemo-immunotherapy regimen by down regulating immunosuppressive factors and increasing lymphocytic infiltration in addition to cell cycle arrest and MEK inhibition. It is yet further believed that MEK inhibition may overcome paclitaxel resistance, which is clinically important to address.

It is yet still further believed that the triple combination treatments of the present disclosure may prolong the median progression-free survival time for a subject having breast cancer (e.g, mBC or mTNBC) as compared to a subject having breast cancer receiving a therapy comprising (i) the therapeutically effective amount of the PD-1 axis inhibitor and the therapeutically effective amount of the MEK inhibitor and without administration of the taxane, (ii) the therapeutically effective amount the PD-1 axis inhibitor and the therapeutically effective amount of the taxane and without administration of the MEK inhibitor, and/or (iii) the therapeutically effective amount the MEK inhibitor and the therapeutically effective amount of the taxane and without administration of the PD-1 axis inhibitor.

### Drug Combination

In some aspects of the present disclosure, a cancer therapy drug combination is provided comprising: (i) a MEK inhibitor in a dose of from about 20 mg to about 100 mg, from about 40 mg to about 80 mg, or about 60 mg; (ii) a PD-1 axis inhibitor in a dose of from about 400 mg to about 1200 mg, from about 600 mg to about 1000 mg, from about 700 mg to about 900 mg, or about 840 mg; and (iii) a taxane in a dose of from about 50 mg/m² to about 200 mg/m², from about 50 mg/m² to about 200 mg/m², from about 50 mg/m² to about 150 mg/m², from about 75 mg/m² to about 125 mg/m², from about 75 mg/m² to about 100 mg/m², about 80 mg/m², or about 100 mg/m², wherein m² is the body surface area of a cancer therapy patient. In one particular aspect, the MEK inhibitor is cobimetinib or a pharmaceutically acceptable salt thereof, the PD-1 axis inhibitor is atezolizumab, and the taxane is paclitaxel or nab-paclitaxel. In some aspects, the combination may be administered every two weeks. For instance, the combination may be administered on days 1 and 15 of a 28-day treatment cycle. In some other aspects, the combination may be administered on day 15 of a 28-day treatment cycle.

In this regard it is to be noted that any combination of the recited dosages ranges for a recited component of the combination may be used without departing from the intended scope of the present disclosure. When a subject is administered the drug combination (i.e., the MEK inhibitor, the PD-1 axis inhibitor and the taxane) on the same day, the drugs may be administered in any order. For instance, (i) the drugs may be administered separately in any order or (ii) a first drug and a second drug may be administered at the same time and a third drug may be administered either before or after administration of the first and second drug. Administration of each drug of the drug combination may be separated by some period of time, such as 0.5 hours, 1 hour, 2 hours, 3 hours or 4 hours. In some particular aspects, cobimetinib or a pharmaceutically acceptable salt thereof may be administered orally, atezolizumab may be administered intravenously, and tpaclitaxel or nab-paclitaxel may administered parentally or intravenously at least 0.5 hours after atezolizumab administration. In such aspects, cobimetinib or a pharmaceutically acceptable salt thereof may be administered before or after atezolizumab. In some aspects, atezolizumab is administered on days 1 and 15 of a 28-day treatment cycle, taxane is administered on days 1, 8 and 15 of a 28-day treatment cycle, and cobimetinib or a pharmaceutically acceptable salt thereof is administered on days 1 to 21 of the 28-day treatment cycle.

### Kits

In some aspects of the disclosure, a kit for treating breast cancer, mBC or mTNBC in a human subject is provided. The kits comprise a MEK inhibitor, a PD-1 axis inhibitor, a taxane and a package insert comprising instructions for using a therapeutically effective amount of the MEK inhibitor, a therapeutically effective amount of the PD-1 axis inhibitor and a therapeutically effective amount of the taxane for treating the subject. In some aspects, the MEK inhibitor is cobimetinib or a pharmaceutically acceptable salt thereof, the PD-1 axis inhibitor atezolizumab, and the taxane is paclitaxel or nab-paclitaxel.

The kits of the present disclosure prolongs the median progression-free survival time as compared to a breast cancer, mBC or mTNBC subject receiving a therapy comprising (i) the therapeutically effective amount of the PD-1 axis inhibitor and the therapeutically effective amount of the MEK inhibitor and without administration of the taxane, (ii) the therapeutically effective amount the PD-1 axis inhibitor and the therapeutically effective amount of the taxane and without administration of the MEK inhibitor, and/or (iii) the therapeutically effective amount the MEK inhibitor and the therapeutically effective amount of the taxane and without administration of the PD-1 axis inhibitor

### EXAMPLES

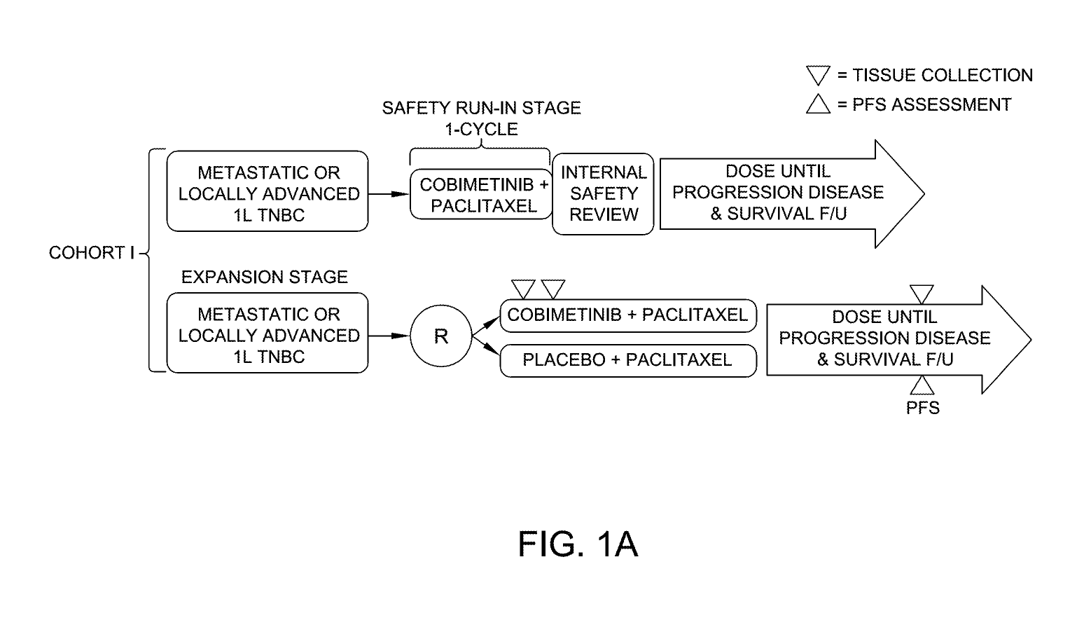

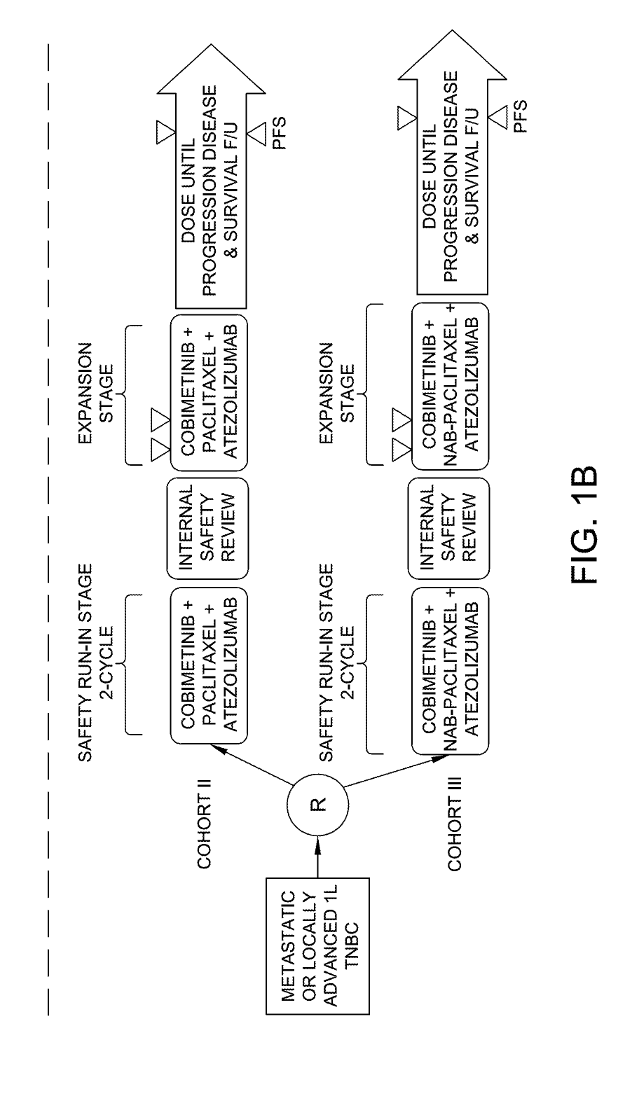

The examples are directed to a three-cohort, multi-stage, randomized, Phase II, double-blind, multicenter, placebo-controlled trial designed to evaluate the safety and tolerability and estimate the efficacy of: (i) cobimetinib fumarate salt and paclitaxel; (ii) cobimetinib fumarate salt, atezolizumab and paclitaxel; and, (iii) cobimetinib fumarate salt, atezolizumab and nab-paclitaxel in patients with metastatic or locally advanced, triple-negative adenocarcinoma of the breast who have not received prior systemic therapy for metabolic breast cancer. FIG. 1A shows the study schema and treatment cohort I and FIG. 1B shows the study schema and treatment cohorts II and III.

Cohort I will investigate the efficacy and safety of cobimetinib plus paclitaxel. Cohort I includes an initial safety run-in stage followed by a randomized (expansion) stage where patients will be randomized to receive either cobimetinib plus paclitaxel or placebo plus paclitaxel.

Following the completion of Cohort I, patients will be randomized (1:1) into either Cohort II or III. Cohort II will investigate the triple combination of cobimetinib, atezolizumab and paclitaxel. Cohort III will investigate the triple combination of cobimetinib, atezolizumab and nab-paclitaxel. Each of Cohorts II and III will comprise a safety run-in stage followed by an expansion stage.

In all treatment cohorts in both the safety run-in and the expansion stages, treatment will be continued until disease progression, unacceptable toxicity, investigator decision, death, withdrawal of consent, or completion of study, whichever occurs first. Because cobimetinib and atezolizumab are investigational agents, for which benefit in this population has not been established, crossover will not be allowed. Tumor measurement for disease evaluation will be performed every two cycles (approximately every 8 weeks). Patients will be monitored throughout the study for adverse events, changes in laboratory values, and physical examination findings. Upon treatment discontinuation, all patients will be followed every 3 months for safety and survival.

Cobimetinib will be administered at a dose of 60 mg on a 21/7 schedule. Cobimetinib (or placebo for patients in the Cohort I expansion stage only) will be taken orally once daily on Day 3 through Day 23 of each 28-day treatment cycle.

Atezolizumab will be administered at a fixed-dose of 840 mg by IV infusion q2w every 14 [±3] days. Preferably, atezolizumab will be administered on Days 1 and 15 of every cycle in Cohorts II and III only.

Paclitaxel will be administered at a dose of 80 mg/m² by IV infusion on Day 1, Day 8, and Day 15 of each 28 day cycle for patients in Cohorts I and II. Because of the known potential for allergic reactions to paclitaxel, patients in Cohorts I and II will be premedicated with dexamethasone, diphenhydramine, and an H2 blocker 30 to 60 minutes prior to the paclitaxel administration and according to the paclitaxel Package Insert and institutional guidelines.

Nab-paclitaxel will be administered according to the local prescribing information. The starting dose level of nab-paclitaxel in this study will be 100 mg/m² administered intravenously over 30 minutes on Days 1, 8, and 15 of each 28-day cycle (3 weeks on/1 week-off schedule).

The dosing scheme is presented in the table 1 below.

TABLE 1
Dosing for Cohort for Each 28-Day Cycle

| Drug | Cohort I | Cohort II | Cohort III |
|---|---|---|---|
| Cobimetinib 60 mg orally once daily for 21 | Days 3 to | Days 3 to | Days 3 to |
| days/placebo (placebo in only applicable to | 23 | 23 | 23 |
| Cohort I expansion stage) | | | |
| Paclitaxel 80 mg/m² intravenous weekly | Days 1, 8 | Days 1, 8 | NA |
| | & 15 | & 15 | |
| Nab-paclitaxel 100 mg/m² intravenous | NA | NA | Days 1, 8 |
| weekly | | | & 15 |
| Atezolizumab 840 mg intravenous every | NA | Days 1 & 15 | Days 1 & 15 |
| two weeks | | | |

The pharmacokinetic analysis population for each drug will include patients who received at least one dose of study drug and provide evaluable pharmacokinetic data. Pharmacokinetic analyses will be conducted for patients with sufficient data to enable estimation of key parameters (e.g., AUC, tₘₐₓ, Cₘₐₓ, t_{1/2}), with patients grouped according to the cohort, stage (safety run in or expansion), and treatment within the expansion stage.

Individual and median plasma cobimetinib, paclitaxel, nab-paclitaxel, and serum atezolizumab concentration versus time data will be tabulated and plotted by drug, cohort, study phase, study visit, and dose level. The plasma or serum pharmacokinetics of cobimetinib, paclitaxel, nab-paclitaxel, and serum pharmacokinetics of atezolizumab will be summarized (such as mean, standard deviation, coefficient of variation [CV %], median, minimum, maximum, geometric mean and geometric mean coefficient of variation [CVb %] as appropriate) for the safety run-in stage (as appropriate for data collected).

mTNBC is a heterogeneous disease, and there are many distinct subtypes of TNBC as defined by molecular signatures (van′t Veer L J, Dai H, Vijver M J, et al., Gene expression profiling predicts clinical outcome of breast cancer, Nature 2002; 415:530-6. Incorporated by reference herein in its entirety.). Therefore, all patients may not be equally likely to benefit from treatment with cobimetinib. Predictive biomarker samples collected prior to dosing will be assessed in an effort to identify those patients with MAPK-driven pathogenesis who are most likely to respond to cobimetinib. Pharmacodynamic biomarkers will be assessed to demonstrate evidence of biologic activity of cobimetinib in combination with paclitaxel in patients and will be assessed in optional on-treatment biopsies which will be collected from patients who consent to this procedure. Disease progression biopsies will be assessed for potential mechanisms of acquired resistance, e.g., emergence of new oncogenic mutations after escaping from the treatment. As these biomarkers may also have prognostic value, their potential association with disease progression will also be explored. In addition to the assessment of PD-L1 status, other exploratory markers such as potential predictive and prognostic markers related to the clinical benefit of atezolizumab plus nab-paclitaxel, tumor immunobiology, mechanisms of resistance, or tumor type, may also be analyzed.

Patient specimens for biomarker analysis will be collected from all patients participating in the trial. These specimens may be used to identify biomarkers that correlate with response/resistance or severity of adverse effects to paclitaxel chemotherapy combined with MEK inhibition. Biomarkers of response and resistance will be identified in clinical specimens collected at pretreatment (archival and/or baseline), during treatment (Cycle 1 Day 15), and at the end-of-study treatment (disease progression). Biomarker analysis may include the following: (A) Expression of oncogenes, tumor suppressors, and genes involved in breast cancer progression to define intrinsic breast cancer subtypes, such as basal subtype, by molecular signatures measured by gene expression analysis; (B) Levels of tumor suppressors (i.e., phosphatase and tensin homolog [PTEN]), expression of immune checkpoints (i.e., PD-L1), mitotic or apoptotic index (i.e., Ki67, Bim, cleaved caspase, or cleaved poly ADP ribose polymerase [PARP]), and immune-cell infiltrations by IHC (i.e., CD8 or FOXP3); and (C) Mutation and copy number changes in oncogenes, tumor suppressors, and/or other genes associated with mTNBC progression by next generation DNA sequencing.

Circulating tumor DNA (ctDNA) can be detected in the blood of cancer patients with epithelial cancers and may have diagnostic and therapeutic significance (Schwarzenbach H, Hoon D S, Pantel K., Cell-free nucleic acids as biomarkers in cancer patients, Nat Rev Cancer 2011; 11(6):426-37. Incorporated by reference herein in its entirety.). For example, the mutational status of tumor cells may be obtained through the isolation of ctDNA (Maheswaran S, Sequist L V, Nagrath S, et al., Detection of mutations in EGFR in circulating lung cancer cells, N Engl J Med 2008; 359(4):366-77. Incorporated by reference herein in its entirety.), and ctDNA has been used to monitor treatment effectiveness in melanoma (Shinozaki M, O'Day S J, Kitago M, et al., Utility of circulating B RAF DNA mutation in serum for monitoring melanoma patients receiving biochemotherapy, Clin Cancer Res 2007; 13:2068-74. Incorporated by reference herein in its entirety.). In accordance with the Examples herein, plasma samples will be assessed for genetic alterations in the MAPK pathway in order to possibly predict which patients may benefit from cobimetinib and to possibly identify potential causes of acquired resistance to cobimetinib. Analysis and correlation of oncogenic mutations in plasma will help to further evaluate the option of using plasma for the detection and monitoring of mutations during the course of treatment.

### Example 1

Example 1 is directed to a Cohort I dose-escalation study for patients treated on a 21/7 schedule with the primary objectives of estimating the maximum tolerated dose (MTD) and clinical benefit, as measured by investigator-assessed PFS, for the combination of cobimetinib and paclitaxel relative to the combination of a placebo and paclitaxel.

Cohort I further includes the following objectives:

Evaluation of the ORR, ORR_uc and DOR of (i) cobimetinib and paclitaxel and (ii) placebo and paclitaxel.

Evaluation of the OS benefit of cobimetinib plus paclitaxel and placebo plus paclitaxel.

Evaluation of the safety and tolerability of cobimetinib administered in combination with paclitaxel. Criteria include measuring the nature, frequency, and severity of adverse effects as graded using NCI CTCAE v4.0. Measured effects include changes in vital signs and clinical laboratory results during and following cobimetinib and paclitaxel administration.

Evaluation of the pharmacokinetics (PK) of cobimetinib and paclitaxel when administered in combination (safety run-in), characterization of PK of cobimetinib and investigation of the relationship between cobimetinib exposure and efficacy and safety outcomes using population approaches (expansion stage). One goal of PK sampling in the safety run-in stages is to check for any differences in cobimetinib and paclitaxel PK when these drugs are co-administered, relative to their PK when administrated alone (historic PK data). The following PK parameters for cobimetinib and paclitaxel will be estimated using data from the safety run-in stage: maximum plasma concentrations (Cmax); minimum plasma concentrations (Cmin); and total exposure (AUC0-τ).

Evaluation of effect cobimetinib and paclitaxel on biomarkers. Evaluations include assessment of the pharmacodynamic effects of cobimetinib and paclitaxel as measured by changes in molecular biomarkers in pretreatment, on treatment, and post treatment tumor tissues. Evaluations further include assessment of the effect of molecular subtypes and genetic alterations on PFS in patients treated with cobimetinib plus paclitaxel versus placebo plus paclitaxel based on analysis of tumor tissue by one or more of the following analyses: (i) intrinsic breast cancer subtypes, such as basal subtype, as defined by molecular signatures measured by gene expression analysis; (ii) mutation and copy number changes in oncogenes, tumor suppressors, and/or other genes associated with mTNBC progression by DNA sequencing; and (iii) levels of tumor suppressors, immune checkpoints, mitotic index, apoptotic index, and/or immune-cell infiltration by IHC. Evaluations further include assessment of the mechanisms of intrinsic and acquired resistances through molecular profiling of tumors prior to treatment and after disease.

Evaluation of the health-related quality of life in patients receiving cobimetinib plus paclitaxel versus placebo plus paclitaxel as measured by European Organisation for Research in Cancer Quality of life questionnaire (“EORTC QLQ-C30”) and Quality of life questionnaire breast cancer module (“QLQ-BR2”). Evaluations will include mean and mean changes from baseline score in all items and subscales of the EORTC QLQ-C30 and QLQ-BR23 by cycle, and between treatment arms.

The Cohort I schedule of pharmacokinetic and anti-therapeutic antibody assessments disclosed in table 2 below will be used:

TABLE 2

| | | | |
|---|---|---|---|
| Visit | Timepoint | Sample Type | Drug |

Cohort I (Cobimetinib plus Paclitaxel) Safety Run In Stage

| | | | |
|---|---|---|---|
| Cycle 1 | Predose | Plasma PK | Cobimetinib |
| Day 8 | | | Paclitaxel |
| Cycle 1 | Predose and 0.5, 1, 2, 4, and | Plasma PK | Cobimetinib |
| Day 15 | 6 hrs postdose | | Paclitaxel |

Cohort I (Cobimetinib plus Paclitaxel) Expansion Stage

| | | | |
|---|---|---|---|
| Cycle 1 | Predose Anytime between | Plasma PK | Cobimetinib |
| Day 15 | 1 and 4 hrs postdose | | |
| Cycle 2 | Predose | Plasma PK | Cobimetinib |
| Day 15 | | | |

### Example 2

Example 2 is directed to a Cohort II study for the triple combination of cobimetinib, atezolizumab and paclitaxel in mTNBC patients.

Cohort II includes the following objectives:

Evaluation of the clinical benefit of cobimetinib, atezolizumab and paclitaxel, as measured by ORR.

Determination of the ORR_uc and DOR of cobimetinib, atezolizumab and paclitaxel, and to evaluate the OS and PFS of cobimetinib, atezolizumab and paclitaxel.

Evaluation of the safety and tolerability of cobimetinib, atezolizumab and paclitaxel. The nature, frequency, and severity of adverse events will be graded using NCI CTCAE v4.0. Changes in vital signs and clinical laboratory results during and following cobimetinib, atezolizumab, and paclitaxel administration with be measured.

Evaluation of the pharmacokinetics of cobimetinib, atezolizumab, and paclitaxel when administered together (safety run in). The pharmacokinetic evaluation in the safety run-in stages will check for any differences in cobimetinib, atezolizumab, and paclitaxel pharmacokinetics when these drugs are co-administered, relative to their pharmacokinetics when administrated alone (historic pharmacokinetic data).

Further, evaluation of the pharmacokinetics of cobimetinib, and investigation of the relationship between cobimetinib exposure and efficacy and safety outcomes using population approaches (expansion stage). The following pharmacokinetic parameters for the combination of cobimetinib, atezolizumab and paclitaxel will be estimated using data from the safety run-in stage: Cmax, Cmin and AUC0-τ.

Evaluation of efficacy objectives by PFS, ORR, DOR and ORR_uc using immune modified RECIST.

Evaluation of the pharmacodynamic effects of cobimetinib, atezolizumab, and paclitaxel as measured by changes in molecular biomarkers in pretreatment, on treatment, and post treatment tumor tissue. Evaluation of the mechanisms of intrinsic and acquired resistances through molecular profiling of tumors prior to treatment and after disease progression. The exploratory outcome measures in archival or baseline, on-treatment, and at progression tumor samples for this study are as follows: (i) intrinsic breast cancer subtypes, such as basal subtype, as defined by molecular signatures measured by gene expression analysis; (ii) mutation and copy number changes in oncogenes, tumor suppressors, and/or other genes associated with mTNBC progression by DNA sequencing; and (iii) levels of tumor suppressors, immune checkpoints, mitotic index, apoptotic index, and immune-cell infiltration by IHC.

Evaluation of any additional treatment burden introduced by atezolizumab as measured by a single item from the physical wellbeing subscale of the FACT-G Quality of Life instrument.

Evaluation of auto-antibodies. For auto-antibody testing, baseline samples will be collected on Cycle 1 Day 1 prior to the first dose of study drug. For patients who show evidence of immune-mediated toxicity, additional samples may be collected. Evaluation includes: anti-nuclear antibody; anti-double-stranded DNA; circulating anti-neutrophil cytoplasmic antibody; and perinuclear anti-neutrophil cytoplasmic antibody.

The following Cohort II schedule of pharmacokinetic and anti-therapeutic antibody assessments disclosed in Table 3 below will be used where “ATA” refers to anti-therapeutic antibodies.

TABLE 3

| | | | |
|---|---|---|---|
| Visit | Timepoint | Sample Type | Drug |

Cobimetinib plus Atezolizumab plus Paclitaxel Safety Run In Stage

| | | | |
|---|---|---|---|
| Cycle 1 Day 1 | Predose | Serum PK | Atezolizumab |
| | 30 min (+/−10 min) post- | Serum ATA | Atezolizumab |
| | atezolizumab dose | (predose only) | |
| Cycle 1 Day 8 | Predose | Plasma PK | Cobimetinib |
| | | | Paclitaxel |
| Cycle 1 Day 15 | Predose and 2 and 4 hrs | Plasma PK | Cobimetinib |
| | postdose | | Paclitaxel |
| Cycle 3 Day 1 | Predose | Serum PK | Atezolizumab |
| | 30 min (±10 min) post- | Serum ATA | Atezolizumab |
| | atezolizumab dose | (predose only) | |
| Cycle 2, 4, 8 and every 8 | Predose | Serum PK | Atezolizumab |
| Cycles thereafter | | Serum ATA | Atezolizumab |
| Treatment discontinuation | At visit | Serum PK | Atezolizumab |
| visit | | Serum ATA | Atezolizumab |
| 120 (±30) days after | At visit | Serum PK | Atezolizumab |
| last dose of atezolizumab | | | |

Cobimetinib plus Atezolizumab plus Paclitaxel Expansion Stage

| | | | |
|---|---|---|---|
| Cycle 1 Day 1 | Predose | Serum PK | Atezolizumab |
| | 30 min (+/−10 min) post- | Serum ATA | Atezolizumab |
| | atezolizumab dose | (predose only) | |
| Cycle 1 Day 15 | Predose | Plasma PK | Cobimetinib |
| | Anytime between 1 and 4 | | |
| | hrs postdose | | |
| Cycle 2 Day 15 | Predose | Plasma PK | Cobimetinib |
| Cycle 3 Day 1 | Predose | Serum PK | Atezolizumab |
| | 30 min (+/−10 min) post- | Serum ATA | Atezolizumab |
| | atezolizumab dose | (predose only) | |
| Cycle 2, 4, 8 and every 8 | Predose | Serum PK | Atezolizumab |
| Cycles thereafter | | Serum ATA | Atezolizumab |
| Treatment discontinuation | At visit | Serum PK | Atezolizumab |
| visit | | Serum ATA | Atezolizumab |
| 120 (±30) days after last dose | At visit | Serum PK | Atezolizumab |
| of atezolizumab | | Serum ATA | Atezolizumab |

### Example 3

Example 3 is directed to a Cohort III study for the triple combination of cobimetinib, atezolizumab and nab-paclitaxel in mTNBC patients.

Cohort III includes the following objectives:

Evaluation of the clinical benefit of cobimetinib plus atezolizumab plus nab-paclitaxel, as measured by ORR.

Determination of the ORR_uc and DOR of cobimetinib, atezolizumab and nab-paclitaxel, and to evaluate the OS and PFS of cobimetinib, atezolizumab and nab-paclitaxel.

Evaluation of the safety and tolerability of cobimetinib, atezolizumab and nab-paclitaxel. The nature, frequency, and severity of adverse events will be graded using NCI CTCAE v4.0. Changes in vital signs and clinical laboratory results during and following cobimetinib, atezolizumab, and nab-paclitaxel administration with be measured.

Evaluation of the PK of cobimetinib, atezolizumab, and nab-paclitaxel when administered together (safety run in). The PK evaluation in the safety run-in stages will check for any differences in cobimetinib, atezolizumab, and nab-paclitaxel pharmacokinetics when these drugs are co-administered, relative to their PK when administrated alone (historic PK data).

Further, evaluation of the PK of cobimetinib, and investigation of the relationship between cobimetinib exposure and efficacy and safety outcomes using population approaches (expansion stage). The following PK parameters for the combination of cobimetinib, atezolizumab and nab-paclitaxel will be estimated using data from the safety run-in stage: Cmax, Cmin and AUC0-τ.

Evaluation of efficacy objectives by PFS, ORR, DOR and ORR_uc using immune modified RECIST.

Evaluation of the pharmacodynamic effects of cobimetinib, atezolizumab, and nab-paclitaxel as measured by changes in molecular biomarkers in pretreatment, on treatment, and post treatment tumor tissue. Evaluation of the mechanisms of intrinsic and acquired resistances through molecular profiling of tumors prior to treatment and after disease progression. The exploratory outcome measures in archival or baseline, on-treatment, and at progression tumor samples for this study are as follows: (i) intrinsic breast cancer subtypes, such as basal subtype, as defined by molecular signatures measured by gene expression analysis; (ii) mutation and copy number changes in oncogenes, tumor suppressors, and/or other genes associated with mTNBC progression by DNA sequencing; and (iii) levels of tumor suppressors, immune checkpoints, mitotic index, apoptotic index, and immune-cell infiltration by IHC.

Evaluation of any additional treatment burden introduced by atezolizumab as measured by a single item from the physical wellbeing subscale of the FACT-G Quality of Life instrument.

Evaluation of auto-antibodies. For auto-antibody testing, baseline samples will be collected on Cycle 1 Day 1 prior to the first dose of study drug. For patients who show evidence of immune-mediated toxicity, additional samples may be collected. Evaluation includes: anti-nuclear antibody; anti-double-stranded DNA; circulating anti-neutrophil cytoplasmic antibody; and perinuclear anti-neutrophil cytoplasmic antibody.

The following Cohort II schedule of PK and anti-therapeutic antibody assessments, disclosed in Table 4, will be used where “ATA” refers to anti-therapeutic antibodies.

TABLE 4

| | | | |
|---|---|---|---|
| Visit | Timepoint | Sample Type | Drug |

Cobimetinib plus Atezolizumab plus Nab-Paclitaxel Safety Run In Stage

| | | | |
|---|---|---|---|
| Cycle 1 Day 1 | Predose | Serum PK | Atezolizumab |
| | 30 min (+/−10 min) post- | Serum ATA | Atezolizumab |
| | atezolizumab dose | (predose only) | |
| Cycle 1 Day 8 | Predose | Plasma PK | Cobimetinib |
| | | | Nab- |
| | | | Paclitaxel |
| Cycle 1 Day 15 | Predose and 2 and 4 hrs | Plasma PK | Cobimetinib |
| | postdose | | Nab- |
| | | | Paclitaxel |
| Cycle 3 Day 1 | Predose | Serum PK | Atezolizumab |
| | 30 min (±10 min) post- | Serum ATA | Atezolizumab |
| | atezolizumab dose | (predose only) | |
| Cycle 2, 4, 8 and every 8 | Predose | Serum PK | Atezolizumab |
| Cycles thereafter | | Serum ATA | Atezolizumab |
| Treatment discontinuation | At visit | Serum PK | Atezolizumab |
| visit | | Serum ATA | Atezolizumab |
| 120 (±30) days after last dose | At visit | Serum PK | Atezolizumab |
| of atezolizumab | | | |

Cobimetinib plus Atezolizumab plus Paclitaxel Expansion Stage

| | | | |
|---|---|---|---|
| Cycle 1 Day 1 | Predose | Serum PK | Atezolizumab |
| | 30 min (+/−10 min) post- | Serum ATA | Atezolizumab |
| | atezolizumab dose | (predose only) | |
| Cycle 1 Day 15 | Predose | Plasma PK | Cobimetinib |
| | Anytime between 1 and 4 | | |
| | hrs postdose | | |
| Cycle 2 Day 15 | Predose | Plasma PK | Cobimetinib |
| Cycle 3 Day 1 | Predose | Serum PK | Atezolizumab |
| | 30 min (+/−10 min) post- | Serum ATA | Atezolizumab |
| | atezolizumab dose | (predose only) | |
| Cycle 2, 4, 8 and every 8 | Predose | Serum PK | Atezolizumab |
| Cycles thereafter | | Serum ATA | Atezolizumab |
| Treatment discontinuation | At visit | Serum PK | Atezolizumab |
| visit | | Serum ATA | Atezolizumab |
| 120 (±30) days after last dose | At visit | Serum PK | Atezolizumab |
| of atezolizumab | | Serum ATA | Atezolizumab |

### Example 4

Table 5 below shows estimated ORR and its 95% CI based on Clopper Pearson method given various observed numbers of responders among the 30 patients in cohort II and III, respectively. Thirty patients provide reasonably reliable estimates for hypothesis generation.

TABLE 5

| Number of Responders | ORR (%) | 95% CI |
|---|---|---|
| 6 | 20 | 7.7-38.6 |
| 9 | 30 | 14.7-49.4 |
| 12 | 40 | 22.7-59.4 |
| 15 | 50 | 31.3-68.7 |
| 18 | 60 | 40.6-77.3 |
| 21 | 70 | 50.6-85.3 |
| 24 | 80 | 61.4-92.3 |
| 27 | 90 | 73.5-97.9 |

This written description uses examples to disclose the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of treating a subject having breast cancer, the method comprising administering to said subject a therapy comprising (i) a therapeutically effective amount of a MEK inhibitor, (ii) a therapeutically effective amount of a PD-1 axis inhibitor, and (iii) a therapeutically effective amount of a taxane.

2. The method of claim 1, wherein the subject has metastatic breast cancer.

3. The method of claim 1, wherein the subject has metastatic triple negative breast cancer.

4. The method of claim 1, wherein the MEK inhibitor is cobimetinib or a pharmaceutically acceptable salt thereof.

5. The method of claim 1, wherein the PD-1 axis inhibitor is a PD-L1 inhibitor.

6. The method of claim 5, wherein the PD-L1 inhibitor is an antibody comprising a heavy chain comprising HVR-H1 sequence of GFTFSDSWIH (SEQ ID NO:24), HVR-H2 sequence of AWISPYGGSTYYADSVKG (SEQ ID NO:25), and HVR-H3 sequence of RHWPGGFDY (SEQ ID NO:12); and a light chain comprising HVR-L1 sequence of RASQDVSTAVA (SEQ ID NO:26), HVR-L2 sequence of SASFLYS (SEQ ID NO:27), and HVR-L3 sequence of QQYLYHPAT (SEQ ID NO:28).

7. The method of claim 5, wherein the PD-L1 inhibitor is an antibody comprising:
(SEQ ID NO: 7)
a heavy chain variable region comprising the amino
acid sequence of
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS
and
(SEQ ID NO: 9)
a light chain variable region comprising the amino
acid sequence of
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

8. The method of claim 1, wherein the PD-1 axis inhibitor is atezolizumab.

9. The method of claim 1, wherein the taxane is paclitaxel or nab-paclitaxel.

10. The method of claim 9, wherein the taxane is paclitaxel.

11. The method of claim 9, wherein the taxane is nab-paclitaxel.

12. The method of claim 1, wherein the subject is treated with from about 20 mg to about 100 mg of the MEK inhibitor per day.

13. The method of claim 1, wherein the MEK inhibitor is cobimetinib or a pharmaceutically acceptable salt thereof, and further wherein the subject is treated with about 60 mg per day of the cobimetinib.

14. The method of claim 1, wherein the MEK inhibitor is administered once daily for 21 consecutive days of a 28-day treatment cycle.

15. The method of claim 14, wherein the MEK inhibitor is administered on days 3 to 23 of the 28-day treatment cycle.

16. The method of claim 1, wherein the subject is treated with from about 400 mg to about 1200 mg of the PD-1 axis inhibitor intravenously every 14 days of a 28-day treatment cycle.

17. The method of claim 16, wherein the PD-1 axis inhibitor is atezolizumab, and further wherein the subject is treated with about 840 mg.

18. The method of claim 16, wherein the subject is treated with the PD-1 axis inhibitor on days 1 and 15 of the 28-day treatment cycle.

19. The method of claim 1, wherein the subject is treated with taxane in an amount of from about 50 mg/m² body surface area to about 200 mg/m² body surface area every 7 days for three weeks of a 28-day treatment cycle.

20. The method of claim 19, wherein the taxane is paclitaxel, and further wherein the subject is treated with about 80 mg paclitaxel/m² body surface area.

21. The method of claim 19, wherein the taxane is nab-paclitaxel, and further wherein the subject is treated with about 100 mg nab-paclitaxel/m² body surface area.

22. The method of claim 19, wherein the subject is treated with the taxane on days 1, 8 and 15 of the 28-day treatment cycle.

23. The method of claim 1, wherein the MEK inhibitor, the PD-1 axis inhibitor and the taxane are each administered on day 15 of a 28-day treatment cycle.

24. The method of claim 1, wherein the PD-1 axis inhibitor and the taxane are each administered on days 1 and 15 of a 28-day treatment cycle and wherein the PD-1 axis inhibitor is administered to the subject prior to administration of the taxane to the subject.

25. The method of claim 1, wherein the taxane is administered before the MEK inhibitor

26. A method of treating a subject having breast cancer, the method comprising administering to said subject a therapy comprising:
(i) a therapeutically effective amount of cobimetinib or a pharmaceutically acceptable salt thereof;
(ii) a therapeutically effective amount of a PD-L1 inhibitor that is an antibody comprising:
(a)
(SEQ ID NO: 24)
a heavy chain comprising HVR-H1 sequence of
GFTFSDSWIH,
(SEQ ID NO: 25)
HVR-H2 sequence of AWISPYGGSTYYADSVKG,
and
(SEQ ID NO: 12)
HVR-H3 sequence of RHWPGGFDY;
and
(SEQ ID NO: 26)
a light chain comprising HVR-L1 sequence of
RASQDVSTAVA,
(SEQ ID NO: 27)
HVR-L2 sequence of SASFLYS,
and
(SEQ ID NO: 28)
HVR-L3 sequence of QQYLYHPAT,
or
(b)
(SEQ ID NO: 7)
a heavy chain variable region comprising the amino
acid sequence of
EVQLVESGGGLVQPGGSLRLSCAASGFTFSDSWIEWVRQAPGKGLEWV
AWISPYGGSTYYADSVKGRFTISADTSKNTAYLQMNSLRAEDTAVYYC
ARRHWPGGFDYWGQGTLVTVSS
and
(SEQ ID NO: 9)
a light chain variable region comprising the amino
acid sequence of
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPAT
FGQGTKVEIKR;
and
(iii) a therapeutically effective amount of a taxane.

27. The method of claim 26, wherein the subject is treated with: about 60 mg of cobimetinib or a pharmaceutically acceptable salt thereof; about 840 mg of the PD-L1 inhibitor; and from about 80 mg/m² body surface area to about 100 mg/m² body surface area of the taxane.

28. The method of claim 26, wherein the taxane is administered before the MEK inhibitor.

29. The method of claim 28, wherein the taxane is administered at least one, two or three days before the MEK inhibitor.

30. A kit for treating breast cancer in a human subject, the kit comprising a MEK inhibitor, a PD-1 axis inhibitor, a taxane and a package insert comprising instructions for using a therapeutically effective amount of the MEK inhibitor, a therapeutically effective amount of the PD-1 axis inhibitor and a therapeutically effective amount of the taxane for treating the subject.

31. The kit of claim 30, wherein the MEK inhibitor is cobimetinib or a pharmaceutically acceptable salt thereof, the PD-1 axis inhibitor is the PD-L1 inhibitor atezolizumab, and the taxane is paclitaxel or nab-paclitaxel.

32. A breast cancer therapy drug combination comprising:
(i) a MEK inhibitor in a dose of from about 20 mg to about 100 mg;
(ii) a PD-1 axis inhibitor in a dose of from about 400 mg to about 1200 mg; and
(iii) a taxane in a dose of from about 50 mg/m² body surface area to about 200 mg/m² body surface area.

33. The breast cancer therapy drug combination of claim 32, wherein the MEK inhibitor is cobimetinib or a pharmaceutically acceptable salt thereof in a dose of about 60 mg, the PD-1 axis inhibitor is the PD-LI inhibitor atezolizumab in a dose of about 840 mg, and the taxane is paclitaxel in a dose of about 80 mg/m² body surface area.

34. The breast cancer therapy drug combination of claim 32, wherein the MEK inhibitor is cobimetinib or a pharmaceutically acceptable salt thereof in a dose of about 60 mg, the PD-1 axis inhibitor is the PD-LI inhibitor atezolizumab in a dose of about 840 mg, and the taxane is nab-paclitaxel in a dose of about 100 mg/m² body surface area.


SEQUENCE LISTING (32 sequences)

SEQ ID NO: 1 - protein, 440 aa, Artificial Sequence
  Synthetic Peptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence
  Synthetic Peptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, Artificial Sequence
  Synthetic Peptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Artificial Sequence
  Synthetic Peptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 117 aa, Artificial Sequence
  Synthetic Peptide
EVQLVESGGGLVQPGGSLRLSCAASGFTFSDSWIHWVRQAPGKGLEWVAWISPYGGSTYYADSVKGRFTSADTSKNTAYLQMNSLRAEDTAVYYCARRHWPGGFDYWGQGTLVTVSA

SEQ ID NO: 6 - protein, 108 aa, Artificial Sequence
  Synthetic Peptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 7 - protein, 118 aa, Artificial Sequence [sequence omitted]

SEQ ID NO: 8 - protein, 122 aa, Artificial Sequence
  Synthetic Peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 9 - protein, 108 aa, Artificial Sequence; identical to SEQ ID NO: 6
  Synthetic Peptide

SEQ ID NO: 10 - protein, 10 aa, Artificial Sequence
  Synthetic Peptide
  MOD_RES (6)..(6): Asp or Gly
GFTFSXSWIH

SEQ ID NO: 11 - protein, 18 aa, Artificial Sequence
  Synthetic Peptide
  MOD_RES (4)..(4): Ser or Leu
  MOD_RES (10)..(10): Thr or Ser
AWIXPYGGSXYYADSVKG

SEQ ID NO: 12 - protein, 9 aa, Artificial Sequence
  Synthetic Peptide
RHWPGGFDY

SEQ ID NO: 13 - protein, 25 aa, Artificial Sequence [sequence omitted]

SEQ ID NO: 14 - protein, 13 aa, Artificial Sequence
  Synthetic Peptide
WVRQAPGKGLEWV

SEQ ID NO: 15 - protein, 32 aa, Artificial Sequence
  Synthetic Peptide
RFTISADTSKNTAYLQMNSLRAEDTAVYYCAR

SEQ ID NO: 16 - protein, 11 aa, Artificial Sequence
  Synthetic Peptide
WGQGTLVTVSA

SEQ ID NO: 17 - protein, 11 aa, Artificial Sequence
  Synthetic Peptide
  MOD_RES (5)..(5): Asp or Val
  MOD_RES (6)..(6): Val or Ile
  MOD_RES (7)..(7): Ser or Asn
  MOD_RES (9)..(9): Ala or Phe
  MOD_RES (10)..(10): Val or Leu
RASQXXXTXXA

SEQ ID NO: 18 - protein, 7 aa, Artificial Sequence
  Synthetic Peptide
  MOD_RES (4)..(4): Phe or Thr
  MOD_RES (6)..(6): Tyr or Ala
SASXLXS

SEQ ID NO: 19 - protein, 9 aa, Artificial Sequence
  Synthetic Peptide
  MOD_RES (3)..(3): Tyr, Gly, Phe, or Ser
  MOD_RES (4)..(4): Leu, Tyr, Phe or Trp
  MOD_RES (5)..(5): Tyr, Asn, Ala, Thr, Gly, Phe, or Ile
  MOD_RES (6)..(6): His, Val, Pro, Thr, or Ile
  MOD_RES (8)..(8): Ala, Trp, Arg, Pro, or Thr
QQXXXXPXT

SEQ ID NO: 20 - protein, 23 aa, Artificial Sequence
  Synthetic Peptide
DIQMTQSPSSLSASVGDRVTITC

SEQ ID NO: 21 - protein, 15 aa, Artificial Sequence
  Synthetic Peptide
WYQQKPGKAPKLLIY

SEQ ID NO: 22 - protein, 32 aa, Artificial Sequence
  Synthetic Peptide
GVPSRFSGSGSGTDFTLTISSLQPEDFATYYC

SEQ ID NO: 23 - protein, 11 aa, Artificial Sequence
  Synthetic Peptide
FGQGTKVEIKR

SEQ ID NO: 24 - protein, 10 aa, Artificial Sequence
  Synthetic Peptide
GFTFSDSWIH

SEQ ID NO: 25 - protein, 18 aa, Artificial Sequence
  Synthetic Peptide
AWISPYGGSTYYADSVKG

SEQ ID NO: 26 - protein, 11 aa, Artificial Sequence
  Synthetic Peptide
RASQDVSTAVA

SEQ ID NO: 27 - protein, 7 aa, Artificial Sequence
  Synthetic Peptide
SASFLYS

SEQ ID NO: 28 - protein, 9 aa, Artificial Sequence
  Synthetic Peptide
QQYLYHPAT

SEQ ID NO: 29 - protein, 118 aa, Artificial Sequence
  Synthetic Peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 30 - protein, 11 aa, Artificial Sequence
  Synthetic Peptide
WGQGTLVTVSS

SEQ ID NO: 31 - protein, 447 aa, Artificial Sequence
  Synthetic Peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 214 aa, Artificial Sequence
  Synthetic Peptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC